(19) United States
(12) Patent Application Publication
(10) Pub. No.: US 2026/0311893 A1
(43) Pub. Date: Oct. 8, 2026

(54) Use of Remote Virtual screening of patients with Artificial Intelligence (AI)with machine learning (ML) capability for initial Screening of the patients and Improving the Efficiency of Medical Diagnosis and Treatment plans.

(71) Applicants: Mammen Thomas, Seattle, WA (US); Arun Mammen Thomas, Dublin, CA (US)

(72) Inventors: Mammen Thomas, Seattle, WA (US); Arun Mammen Thomas, Dublin, CA (US)

(21) Appl. No.: 19/656,825

(22) Filed: Apr. 23, 2026

Related U.S. Application Data
(63) Continuation-in-part of application No. 19/179,944, filed on Apr. 15, 2025.
Application No. 19/179,944 is a division of application No. 18/930,973, filed on Oct. 29, 2024, now Pat. No. 12,437,881.

(51) Int. Cl. G16H 80/00; G06F 40/58; G06T 17/00; G16H 10/20; G16H 50/20

(52) U.S. Cl. CPC G16H 80/00; G06F 40/58; G06T 17/00; G16H 10/20; G16H 50/20

(57) ABSTRACT

A method and process for providing virtual screening of patients implemented as a virtual universe with the help of a remote system having Artificial-Intelligence with machine-learning capability for collection and presentation of patient's personal and medical data is provided. Currently Doctors conduct virtual visits with patients to help collect patient's data and establish initial diagnosis. This is a time taking process and takes up a lot of the physician's and their assistant's time and energy. Using the remote system to do the initial screening and collection of patients data for the physician before the virtual visit, will reduce the time for the physicians to arrive at a diagnosis. The capability will also help a physician to guide an assistant at a remote location to collect additional data of the patient, by interacting with the assistant and the patient using the 3-D communication channel or as avatars in the virtual reality.

### PRIOR APPLICATION DETAILS

This application is a continuation in part of patent application Ser. No. 19/179,944 a division of patent application Ser. No. 18/930,973 both titled: “Use of Remote Virtual Screening of Patients with Artificial Intelligence (AI) and Machine Learning (ML) capability for initial Screening of the Patients for Improving the Efficiency of Medical Diagnosis and Generation of Treatment Plans”.

### FIELD OF INVENTION

The invention relates to improving interaction between a patient at remote location and a medical team for diagnosis and identification of treatment options, especially the screening of patients in remote locations using the capabilities of Artificial intelligence, Machine learning with natural language recognition and then connecting to medical teams over internet or as avatars, using preferably three dimensional (3-D) virtual image of patients preferably implemented as a virtual universe, where the initial screening of a patient can be done by the AI using natural language.

### DESCRIPTION OF FIGURES

FIG. 1, (100) is a depiction of an embodiment of a system of the present invention enabling a Primary clinic doctor interacting with a patient at remote site over internet using 3-D image on screen or within a virtual reality environment.

FIG. 2, (200) is an exemplary natural language understanding and question generation system with AI and ML capability for understanding the patient's problem and generating an initial diagnosis.

FIG. 3, (300) is an exemplary flow chart of the use of the system having AI with ML capability to generate the initial diagnosis without involving the doctors.

FIG. 3A (300A) is an exemplary flow chart of the capability developed as an embodiment of the current invention for remote doctor patient interaction either via high resolution 3-D images or within a virtual reality environment to finalize a diagnosis and treatment plan with follow-up.

FIG. 4, (400) is a typical diagram of an artificial intelligence (AI) with machine learning (ML) capability capable of using large language models (LLM) showing its components used in the disclosed system for generating the report comprising the personal and Medical history of an individual patient with a preliminary diagnosis and treatment plan.

FIG. 5, (500) is an architectural block diagram of the exemplary Agentic AI with ML capability, similar to Avirteck, that may be used in the system shown.

FIG. 6. (600) is a block diagram of the distributed data processing and storage system that may be used with an embodiment of the disclosure.

### SUMMARY

Embodiments of the invention provide for a system for allowing interaction between a doctor and a patient at a remote location, the system comprising a capability to generate 3-D image data of a doctor's office and a 3-D image data of a remote patient location using sensors, wherein the generated image data are used to create a 3-D projection or a virtual projection or a virtual environment of metaverse enabling the interaction of the doctor and the patient. Embodiments of the invention provide for a system where the patient at the remote location and the doctor are able to communicate with each other over the internet or within the virtual environment of metaverse enabling the interaction of the doctor and the patient.

Embodiments of the invention provide for a system where the patient and doctors are able to communicate with each other over the internet or within the virtual environment of metaverse. Embodiments of the invention are directed to interacting with a patient at a remote location using a system capable of receiving natural language inputs and converting that to a medically understandable language using external conversational data inputs and stored language inputs into a language understanding system which has an in-built artificial intelligence (AI) and a machine learning (ML) capability to provide the natural language understanding and conversion capability.

The system uses artificial Intelligence (AI) with ML capability to generate relevant questions to extract from the patient data needed to arrive at an understanding of the patient's problem.

The system saves the list of questions and answers in a database for enhancing the machine learning capability of the system and to use with the artificial intelligence. to enable the system to optimize future interactions with patients.

The system creates a clinical knowledge database that is generated and continually updated from various medical sources external to the system.

The system uses the clinical knowledge database with the patients answers and the saved answers to arrive at an initial diagnosis for the patient using an AI based Automatic diagnosis unit.

The AI based automatic diagnosis unit also generates an initial treatment plan for the patient based on the initial diagnosis and input from the clinical knowledge database.

Once the initial assessment, comprising initial diagnosis and initial treatment plan, is done the system sends the diagnosis and treatment plan over a communication channel to doctors at a primary clinic for review and further action.

The system uses the sensors covering the patient's room at the remote location and the doctor's office at the primary clinic enable the system to generate image data to provide a high resolution 3-D image for interaction between the doctors and the patient as 3-D images on screen or as a 3-D projection, or in an environment of virtual universe enabling the interaction of the doctors with the patient as their avatars.

The system enables the doctor at the primary clinic to interact with the patient at the remote location directly using a 3-D interactive Video, a 3-D projection or within a virtual environment, to ask further questions and receive answers to refine the diagnosis and arrive at an optimized diagnosis of the patient's current problem.

The system also allows the doctors to discuss with the patient and arrive at a treatment option and a follow up option that the patient is comfortable with based on the final diagnosis.

The use of such a system enable virtual doctor patient interaction for putting the patient diagnosis and treatment plan in place while most of the interaction and time spent till the development of the initial diagnosis and treatment plan is handled by the natural language understanding and question generation system with AI with ML capability.

The system by reducing the time the doctors have to spend with each patient to understand and arrive at a preliminary diagnosis, improves the efficiency of care.

### DETAILED DESCRIPTION

In many regions of the world access to medical facilities for diagnosis and treatment of diseases is very limited. For example, in Australia the flying doctors have to be sent remote locations by aircraft with the associated cost and time to check and diagnose medical problems. In some locations in Africa, India and in some cases in the US, the access to and availability of doctors at remote locations is limited. This has created a need for an ability to be established that allow minimally trained doctor's assistants in remote locations, who are local and able to connect to the patients, to virtually connect patients to extract vital details and to interact with consultants and specialists, when the need arises. A method and process for providing virtual screening, by video or three dimensional (3-D) virtual image of patients implemented as a virtual universe, for medical practitioners to interact with the patients, over the internet or as avatars in the virtual universe, for collection of current and historic medical data, diagnosis and treatment, is disclosed. Further disclosed is the capability to use an artificial intelligence (AI) with machine learning (ML) capability (AI with ML capability hereinafter) to collect routine data and data on patient's current problem, from the patient, with or without help from any available minimally trained doctor's assistants, to reduce the load on the trained medical staff and doctors. Doctors are further enabled to conduct virtual visits with patients to help collect any additional medical data, establish an initial diagnosis and interacting with the patient create a treatment plan for the presented problem of the patient. The collection of patients current and historical data is the most time taking process and takes up a major portion of the Doctor's and their assistant's time and energy during any visit. Using the AI with ML capability to do the initial screening and collection of patient's non-specific and specific medical data to be presented to the medical team before the virtual visit with the patient, will reduce the time the physicians and their team need to spend with the patient during the virtual visit. AI with ML capability can also be used to arrive at the initial diagnosis and the treatment plan to be provided to the medical team. The capability will also help minimally trained doctor's assistants and bare foot doctors at the remote clinics, at remote locations, to collect additional data of the patient by requesting clarification of the patients answers where required. Since the number of physicians and time available for the physician/physician assistants are a premium in these remote location for \diagnosis efforts, having an AI robot with ML capability to collect and present the routine and specific medical information of patients will be of great import in hard to reach places and where medical facilities are difficult to get to. The AI robot with ML capability will be able to interact with the patient at the remote clinic to collect the initial patient information such as weight, height, oxygen saturation of blood, blood pressure etc. if the AI with ML capability is programmed to set up the needed instrumentation for doing these tasks, with or without the help of the minimally trained doctor's assistants, at the remote clinic. Further if the AI with ML capability is enabled with natural language understanding and translation capability, the AI with ML capability can generate and ask questions to the patient and accept the natural language inputs from the patient. From these natural language interactions, the AI with ML capability will be able to identify medically relevant terminologies and their association within the natural language inputs. The AI with ML capability will also be able to screen the patient, by asking a series of inter linked questions and collecting the answers, to identify what kind of problem the patient has come to the doctor with. The AI with ML capability can then use the patient's responses and clarification answers to extract medically relevant data regarding the patient to generate the medical history of the patient and present it to the medical assistant or the medical team at the clinic. The medical history and the presented problem enabling the medical assistant, nurse practitioner or the physician to decide to collect additional data, arrive at an action plan for additional investigation and the next steps of treatment for the patient without spending too much time and effort up front with the patient, collecting current and historical medical data of the patient. The presentation itself can be made over the Video over the internet or as avatars in the virtual universe, where the physician and assistants can directly interact with the patient, for collection of additional medical data, establishing a final diagnosis and generating the treatment plan for the patient.

One of the advantages of using the AI with ML capability is the unique ability of the AI with ML capability to refine the initial interaction and the diagnostic capabilities as more and more interactions with patients and medical team happen.

Though the current application is focused on the use of AI with ML capability for remote applications it is not to be considered limiting. AI with ML capability as developed can also be used in Doctor's offices to reduce the load on the nurses and Nurse Practitioners.

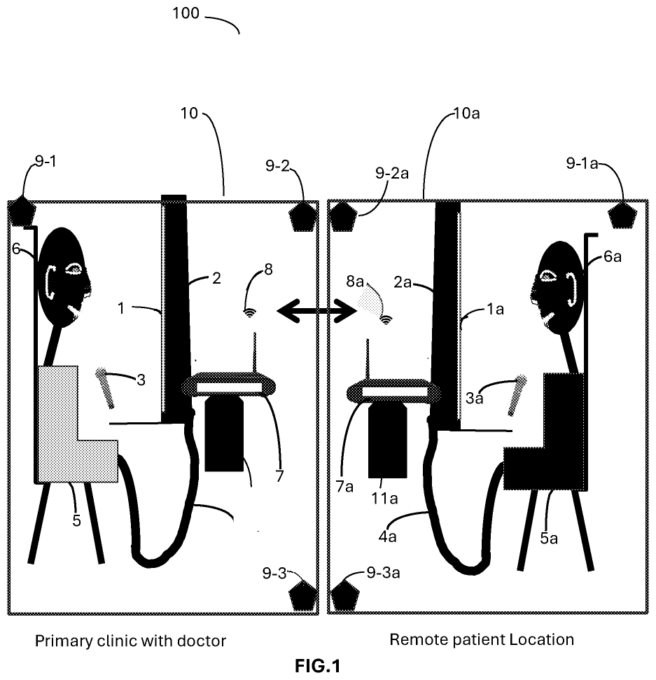

FIG. 1, 100 shows a nonlimiting system embodiment of the invention using a typical setup for the interaction of a doctor 6 with a remote patient 6a. The Set up comprising a doctor's room 10 having sensors 9-1 to 9-3 for generating an image of the volume of the room for use in a virtual reality environment when such an engagement is required, a console chair 5 for the physician to use have a built in processing capability, that is a grouped processors and GPUs interconnected to provide sufficient processing capability for all the applications including the AI with ML capability that typically uses large language models for training the AI, the physician having a 3-D display terminal 1 with a console circuit box 2 and a microphone 3. The sensors 9-1 to 9-3 are linked to the console circuit box and the processing capability built into the console box at the chair with the AI with ML capability 11 to integrate the collected data for display in the virtual reality environment and use natural language for interaction. The connection between the console circuit box and the console under the chair 5 having the processing capability is shown as wired 4. The connection to the remote patient 6a is shown as over an internet connection 7 to 7a (shown over a wireless connection 8 to 8a, This connection is not to be considered limiting as a wired connection is also applicable) connecting a remote set up for the patient 6a at a location that is similar to that of the doctor 6, the set up comprising a patient examination room 10a with sensors 9-1a to 9-3a coupled to the condole circuit box 2a and the AI with ML capability 11a. The patient also sits on a console chair 5a having a grouped processing capability comprising interconnected processors and GPUs. The processing capability being sufficient to handle all the applications including the AI with ML capability wherein the AI is typically trained on Large language models. The patient sitting on the console chair 5a is in front of a 3-D display terminal 1a with a consol circuit box 2a and a microphone 3a. The processing capability is coupled to the console circuit box 2a and the AI with ML capability 11a using the wired connection 4a. It is to be specifically understood that the setup in FIG. 1 (100) is provided as an example only and the final setup will depend on the design of the facility at the doctor's office and the remote patient location.

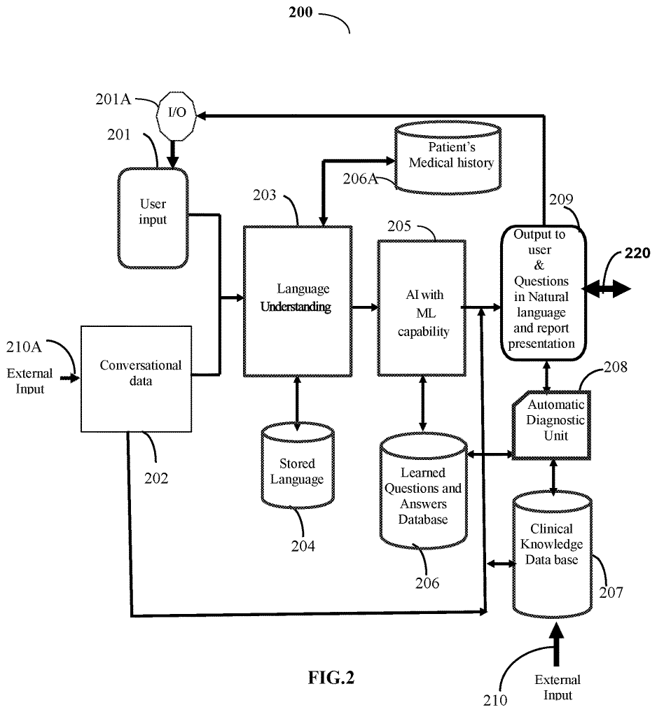

FIG. 2, 200 is an exemplary natural language understanding and question generation system with AI and ML capability. The AI with ML capability is used for understanding the patient's problem and generating an initial diagnosis and an associated treatment plan. The user input is via the input output I/O module 201A comprising the microphone 3a in FIG. 1. The input received is shown as incoming input and comprise the personal information, the medical history and the patient's current medical Problem, 201, which is then combined with the conversational language database, 202 supplemented by external input, 210A and previously stored language data from the stored language database 204 are used to convert the natural language input to medically understandable terminology to arrive at an understanding of the Patient's personal data, medical history and the nature of the patient's current medical problem at the language understanding block, 203. The natural language understood and converted medical terminology is also used to update the database, 204 for future use. The understood inputs with language stored in the database 204 is used by the AI with ML capability 205 to generate additional questions to be presented to the patient in natural language from the user output module shown as the output questions in natural language block, 209 via the I/O module 201A. The AI with ML capability,205 generates questions to be put to the patient. The answers to these questions are used to refine and optimize the understanding of the patient's Personal information and the patient's medical problem. The questions and answers are further used to train the AI with ML capability, 205 to improve the functionality of patient interactions, using the available user inputs, 201 from patients and the previously generated questions and answers saved in a learned questions and answers database, 206. The AI with ML capability, 205 generated sets of questions and answers are also used to update the stored data in the Learned questions and answers database, 206 for providing input into the AI with ML capability, 205 as updated patient information contained in the updated the Learned Questions and Answers database.

A clinical knowledge database 207, that is constantly updated with externally available clinical knowledge data via an external input, 210 is used to provide up to date medical knowledge to the system, 200. database, 206 enable the Automatic diagnosis unit enabled with AI, 208 to generate and provide an initial diagnosis and treatment plan for the patient.

The link 220 is used to send the generated report to the medical staff and doctors.

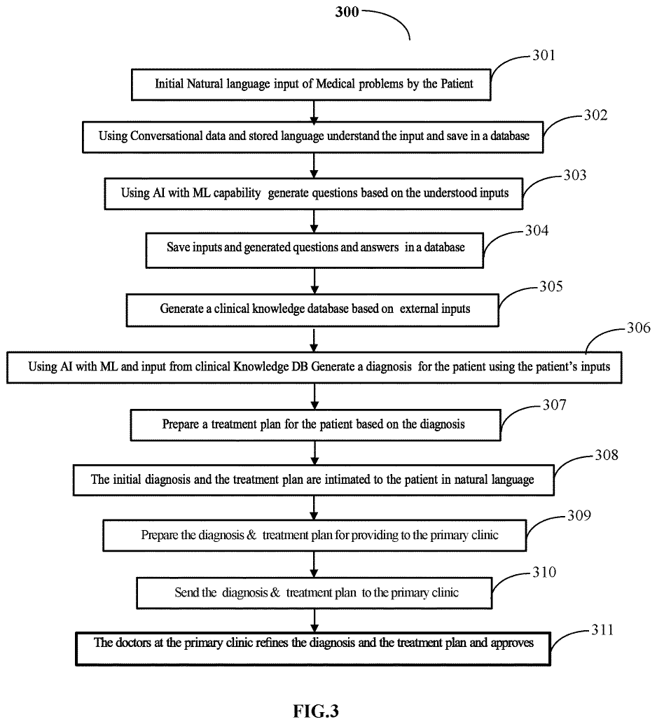

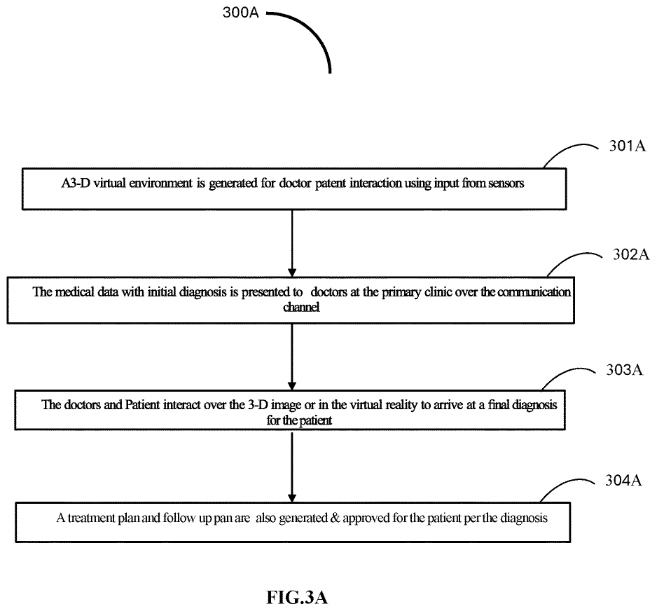

FIG. 3, 300 is a simplified exemplary flow chart of the operation of the system in FIG. 2 to arrive at an initial diagnosis and a treatment plan for the patient without involving a trained physician or physician assistant.

The patient arrives at the remote location 10a and is guided by a technician to the console seat 5a with the voice input 3a and visual screen and console circuit 2a. The patient then describes his personal information and the symptoms of his problem in natural language into the system, at step 301. Any new personal information and Medical history presented by the patient is used to update the information in the Patient's Medical History and information database 206A. This information may be used to prepare a report to the doctors at the primary clinic.

Using the available conversational language data from the external input 10a combined with previously stored language information in the stored language data base 204, the language understanding block 203 prepares the natural language input into a medically understandable input to be provided to the AI with ML capability 11a shown in FIG. 1 and the system block 205 of FIG. 2, at step 302.

The AI with ML capability is used to learn the medical conversion of the natural language inputs as they are presented to the AI with ML capability system block to refine the understanding of the inputs and generate additional questions to be presented to the patient to elucidate his problem for further understanding by the AI, at step 303.

The available inputs, generated questions and answers are saved and updated within a Learned questions and answers data base, 206 constantly as new patients interact with the system. The data in the database is used with the inputs from patients by the AI to improve and refine the questions generated for the patient to clarify the problem presented by the patient. The generated questions are converted to natural conversational language and presented to the patient using the visual screen and speakers available on the console circuit 2a at the remote location, for the patient to provide answer, at step 304.

A continuously updating database of clinical knowledge ,207 is built and kept updated with external inputs and the knowledge base is made available to the system, at step 305.

Using the clinical knowledge base and the understood inputs from the questions and answers between the patient and the system saved in the database, 206, the AI based Automatic Diagnosis unit, 208 generates an initial diagnosis for the patient, at step 306.

Based on the initial diagnosis the AI based Diagnosis unit also generates a treatment plan for the patient, at step 307.

The initial diagnosis and the treatment pan are made available to the patient, at step 308.

The initial diagnosis and the treatment plan are made ready for transfer to the doctors at the primary clinic, at step 309.

The doctors can the approve the diagnosis and treatment plan or connect to the patient over the network to ask further clarifications to the patient and refine the diagnosis and treatment plan at step 310.

FIGS. 3A, 300A is an exemplary flow chart of the interaction between the doctors at the primary clinic and the patient at the remote location after they have received the initial diagnosis and the treatment plan developed by the AI based diagnosis unit. At the remote location.

A 3-D image or a virtual environment in metaverse is generated with the patient sitting on the console chair 5a in front of the display console circuit 2a with the microphone 3a at the remote location and the doctor at the primary clinic on in front of the console with the communication capability 5 using the 3-D data collected by the sensors 9-1 to 9-3 and 9a-1 to 9a-3 at the two locations. The system enable the doctors to interact with the patient in the virtual environment of metaverse or 3-D display. at step 301A.

The diagnosis and treatment plan developed automatically using the remote system having AI with ML capability is sent over the internet connection and presented to the doctors at the primary clinic for review, at step 302A.

The doctors at the primary clinic interact with the patient to refine the initial diagnosis by asking additional questions and explain the final diagnosis and the treatment plan to the patient over the 3-D display or within the virtual environment of the metaverse, at step 303A.

A diagnosis and a treatment plan as well as plan for follow-up and review are agreed between the patient and the doctor during the interaction, at step 304A.

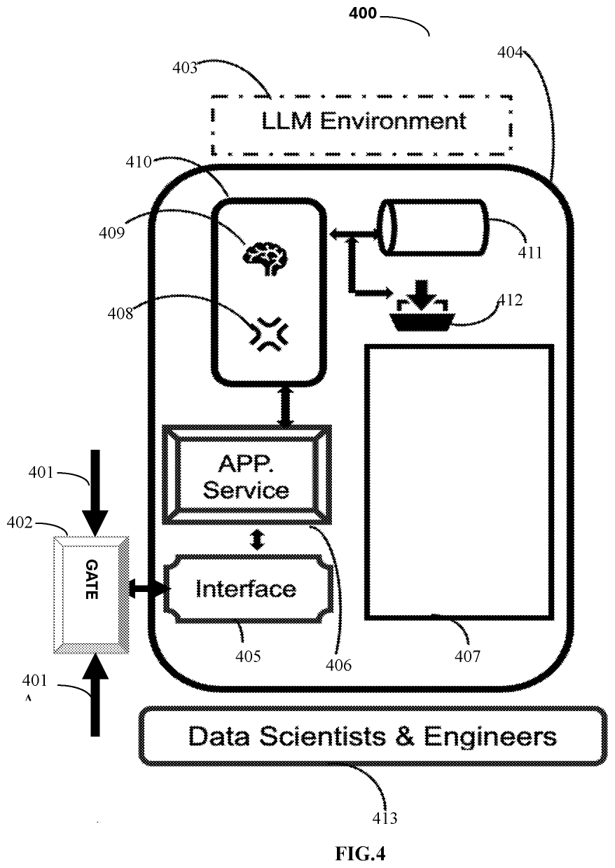

FIGS. 4, 400 is an exemplary block schematic of an AI with ML capability. The data gate way, 402, enable data, 401, from all data sources and user inputs and outputs, 401A, to be screened for unwanted and malicious cyber-attack components. The screened data is then allowed to enter the protected large language (LLM) environment, 403, comprising the protected modules of the AI with ML capability, 404, via the interface 405. The protected AI with ML capability, 407, may in certain cases allow pre-screened Data scientists and Engineers 405, direct access. The interface, 405, may include an application programming interface (API) in some embodiments. The protected LLM environment 403 comprising the protected AI with ML capability, 404, may comprise an applications(APP) service module, 406, that couples to the specific application modules within the system such as the input/output (I/O) module, 201A, The user input module ,201, language understanding module, 203, the user output for questions in natural language module, 209, the automatic diagnostic unit, 208, etc. of the system 200, an AI production service module, 410, that may comprise the language and AI models 408 and language and AI agents, 409, of the agentic AI used. The protected AI with ML capability, 404 may also comprise an AI training data base, 411, for storing training data extracted from the various data bases in the system. The AI training data base, 411 is coupled to the second console, 10, in the disclosed system in FIG. 1 that comprise all the data bases (DB)s such as the conversational DB, 202, the stored language DB, 204, learned questions and answers DB, 206A, learned diagnosis and treatment plan DB, 206B, the clinical knowledge DB, 207, etc. A retrieval augmented generation (RAG) module 412 enable the results from the AI core application service module, 407, working in conjunction with the system APPs coupled to the APP service, 406, to be evaluated to provide up-to-date, accurate answers without needing to retrain the models used. The use of RAG enable the system to provide outputs that are current with data from the continuously updated conversational data DB, 202, updated with external input ,211A, and clinical knowledge DB, 207, updated from external sources via input, 211B.

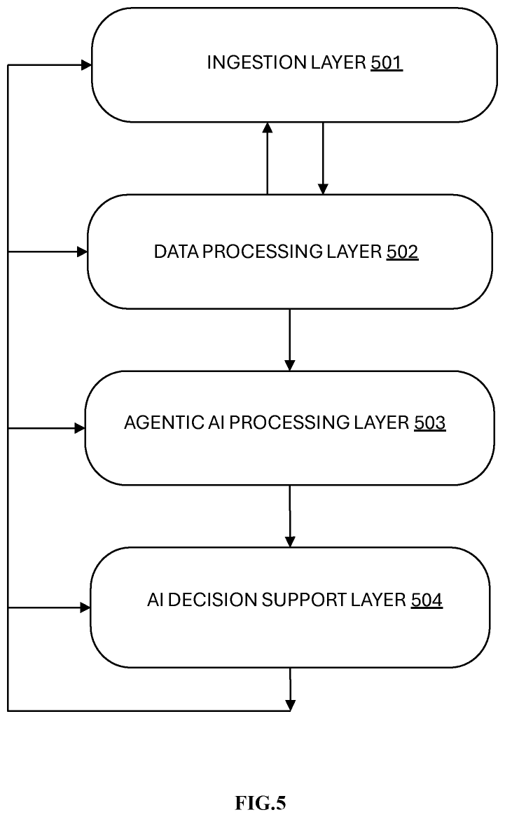

FIGS. 5, 500, is an architectural block diagram of the exemplary agentic AI with ML capability. There are four main blocks defining the architecture of a typical agentic AI with ML capability. They are the data ingestion layer, 501, the data processing layer, 502, the agentic AI processing layer,503, and the AI decision and decision support layer, 504.

The data ingestion layer, 501, handles all incoming data from various data sources in multiple formats, it accepts data via the connection, 401, from the external network and data bases. The data may be in the form of videos, voice, pictures and structured or unstructured text, in multiple formats. The data ingestion layer, 501, accepts these inputs in multiple types and formats and prepares them to a form acceptable to the system. The input from external sources in the current case includes but are not limited to the continuous update, 211A, into the conversational database 202 mainly used by the language understanding module,203, and the user output for questions in natural language module, 209 and the continuous update, 211B, into the clinical knowledge base database, 207 mainly used by the automatic diagnosis module, 208, that develops the preliminary diagnosis and the preliminary treatment plan.

The data Processing layer 502 functions to perform the classification of the data into data types such as video, audio, image or text. It also detects the data formats and converts the data into standardized format that is usable by the large language models environment (LLM Environment) , 403, and the AI with ML capability, 404, that forms within that environment. The data processing layer also comprises the specialized AI models such as Video expert, Image expert, Audio expert, speech and language expert etc. to be used with the data received. The data processing layer also has control over the vector data base within the system that is powered by retrieval-augmented generation (RAG), 412,capability within the AI, 404, that is used for achieving selected knowledge storage and retrieval for use by the AI, 404 that is part of the AI module, 205, in the system disclosed.

The agentic AI processing layer 503 handles all the operations of the domain expert AI components of the data processing layer. This layer uses interaction between multiple AI experts working in conjunction with modules 203 and 209 to generate intelligent questions and intelligent responses, eliminating inaccurate responses. This layer providers the questions to be put to the patient in natural language to have his responses that allows collection and clarification of his personal information and medical history as well as to clearly understand his current medical problem. The agentic AI processing layer also prepares and reviews the output of the AI operating with the Automatic diagnostic module, 208, and to generate the preliminary diagnosis and the preliminary treatment plan for the patient and put together the report to be sent to the medical professionals/doctors over the communication channel provided by the module, 212, of the system, 200.

The AI decision layer, 504, has over all control of the process within the LLM environment 403 comprising the AI with ML capability unit, 404. It validates all responses output by the AI environment, 404. It receives input from the system components that allows it to have an accurate and consonant control over the current status and respond accurately operational questions and corrective actions to be taken in case of problems with the system. This section is mainly in contact with the engineers and data scientists, 405, who have direct access to the operating AI environment, 404, to correct any system errors.

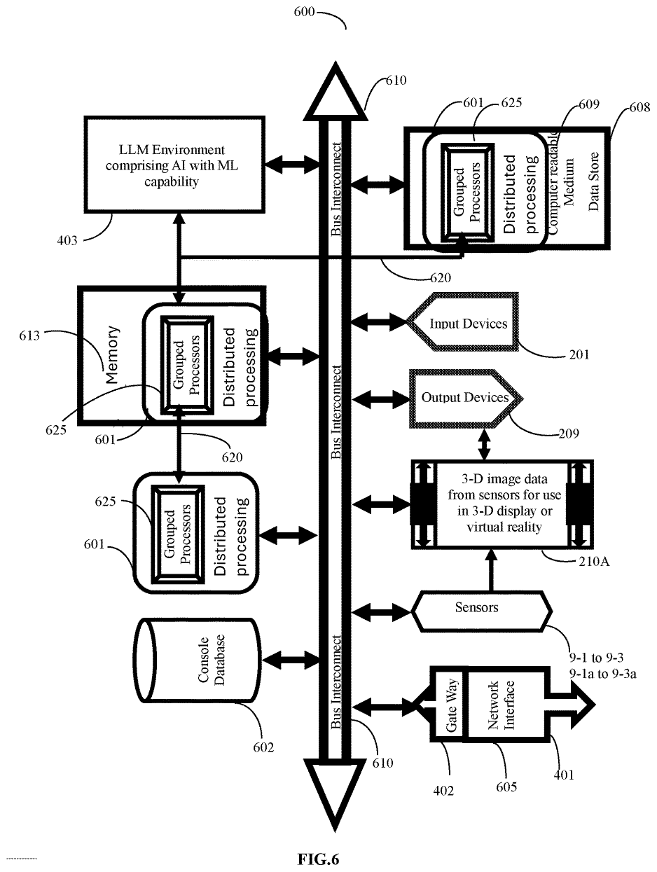

FIGS. 6, 600, is a block diagram illustrating an example of a data processing system which may be used in the patient' location and the doctor's office location with an embodiment of the disclosure. For example, system 600 may also include representation of the system 200 described above performing any of the processes or methods described. System shown in 600 can include many different components. These components can be implemented as integrated circuits (ICs), portions thereof, discrete electronic devices, or other modules adapted to a circuit board. The data processing system 600 may have many components in addition to those shown in different implementations, furthermore it should be understood that the arrangement of components may differ in different implementations. While only a single system is illustrated the term machine or system used is not to be construed as limiting to one single system or machine. The terms may describe a plurality of systems or machines that individually or jointly execute a set (or multiple sets) of instructions to perform any one or more of the methodologies discussed. The embodiment shown has distributed processing capability, 601, each with one or more processors, where if a plurality of processors are used they are grouped together to form grouped processors, 625, the distributed processing capabilities are linked or coupled together by links 620 to create a large processing group capability with sufficient processing power to handle the massive data inputs and tasks assigned to the system. The link, 620, may comprise PCI links interconnecting switches that enable processors to work together, in groups within the processing capability. By interconnecting these switches, a larger interconnected group with large, shared processing capability may be established for the system, 600. Processing capability may include processors 601 that may represent one or more general-purpose processors such as a microprocessor, a central processing unit (CPU), or the like. More particularly, processor, 601, may be a complex instruction set computing (CISC) microprocessor, reduced instruction set computing (RISC) microprocessor, very long instruction word (VLIW) microprocessor, or processor implementing other instruction sets, or processors implementing a combination of instruction sets. Processor 601 may also be one or more special-purpose processors such as an application specific integrated circuit (ASIC), a cellular or baseband processor, a field programmable gate array (FPGA), a digital signal processor (DSP), a network processor, a graphics processor, a communications processor, a cryptographic processor, a co-processor, an embedded processor, AI processor, or any other type of logic capable of processing instructions.

The system shown has various other components in addition to the processing capability that include the data bases in the console 602 shown as the database console, 10 of (FIG. 1) of the system diagram 100, the network interface, 605, and gate way, 402, that allows external data access, the input/output module 201A comprise the linked input devices,201, and the linked output devices of module, 209 and the output to the doctors, 220, of the system disclosed for accepting natural language inputs and video input from the patient, comprising the video sensors/camera and audio inputs, the output devices, 209 for natural language out pus of generated questions to the patient. A 3-D image data generating module 210A, coupled to the sensors 9-1a to 9-3a in the patient's room location and the sensors 9-1 to 9-3 in the doctor's office location generate the 3-D image data for enabling the interaction between the doctor(s) and the patient on the web or as Avatars in the virtual environment described. The 3-D image data may be transferred wirelessly as shown in FIGS. 1,8 or over link 220 in FIG. 2 or 401 in FIG. 6. The operating memory, 603, the computer readable medium,609, and computer storage 608 and the LLM environment, 403, comprising the AI with ML capability, 404 of FIG. 4. All the components of the system are inter linked on a common bus, 610, for sharing the data available during operation.

The system, 600, may comprise additional input and output modules that in some embodiments may be used by the engineers and data scientists, 413, to monitor the functionality of the system and interact with the AI decision support layer 504 to understand the system status and initiate any corrective action necessary.

The computer readable medium 609 may contain software that when executed will cause the components/modules of the system to operate together to provide the functionality described.

The system as described is able to off load the operation of collecting patient's personal and medical data and also defining tests to arrive at an initial diagnosis for the patient. By generating a preliminary report comprising these to be provided to the doctors and medical personal, the system is able to reduce the time spent by the doctors and other medical personal. By of lading even a portion of the load and time spent (time pressure) on the doctors and other medical staff spent for collection of data and report preparation will enable the doctors and medical personal more time to cater to the patients and improve the efficiency of the clinical set up. This will also improve the quality of care by improving the utilization of scares resources and improve their efficiency of patient care.

Even though the inventive idea is described here using a specific embodiment, it is not meant to be limiting. There will be many changes that can be implemented to the embodiment that will achieve the same result as will be well known to practitioners of the art. These are covered by the current application.

### From Chat ZGPT Support for Data Collection Load on Doctors

“A study revealed that physicians spend 49% of their time on Electronic Health Records and other desk work, and only 27% on direct clinical face time with patients. This means about half their work is related to data collection and management, and a quarter to direct patient interaction.”

(The balance 24% is spent in management.)

1. A system that interacts with a patient at a remote location, the system comprising an input/output (I/O) module coupled to a user input module to receive natural language inputs and convert that to a medically understandable language, using conversational data from a continuously up dating conversational data database (DB) and previously stored language inputs from a stored language DB, in a natural language understanding module.

2. The system of claim 1, wherein the natural language understanding module is coupled to an artificial intelligence (AI) with a machine learning (ML) capability for generating additional questions to be presented to the patient in natural language via the I/O module coupled to an output to user, questions in natural language presentation module.

3. The system of claim 2, wherein an answers to the questions by the patient is used to update and understand the patient's current medical issues.

4. The system of claim 3, wherein the patient's input is also used to update the patient's personal information and the patient's medical history.

5. The system of claim 4, wherein the patients personal and medical history and an understanding of the patient's current medical issues are used to generate a preliminary diagnosis and a preliminary treatment plan for the patient using inputs from a continuously updating clinical knowledge DB.

6. The system of claim 3, wherein the system saves the list of questions generated by the AI with ML capability and updates the question and answers DB for enhancing the machine learning capability of the system and for use with the AI based automatic diagnostic unit; the updated data in the question and answers DB is further used by the system to optimize future interactions with patients.

7. The system of claim 5, wherein the clinical knowledge data base that is continuously updated is form medical data from multiple external medical sources available.

8. The system of clam 7, wherein the data in the clinical knowledge DB is also used to generate and present questions to the patient.

9. The system of claim 4, wherein the data in the clinical knowledge data base and the data in the stored question and answers data base is used by the AI coupled to an automatic diagnosis unit to arrive at an initial diagnosis for the patient.

10. The system of claim 9, wherein the automatic diagnosis unit also generates an initial treatment plan for the patient based on the initial diagnosis arrived at and input from the clinical knowledge data base.

11. The system of claim 10, wherein the system sends the initial diagnosis and the generated initial treatment plan over a communication channel to doctors and medical professionals at a primary clinic for review and further action.

12. The system of claim 11, wherein the review of the initial diagnosis and the initial treatment plan is confirmed or updated by the doctors and medical staff at the primary clinic by the doctors or medical staff at the primary clinic as their avatars discussing and interacting with the patient also as an avatar in a virtual reality environment.

13. The system of claim 11, wherein the review of the initial diagnosis and the initial treatment plan is confirmed or updated by the doctors and medical staff at the primary clinic by the doctors or medical staff at the primary clinic discussing and interacting with the patient over 3-D displays available at the remote patient location and the primary clinic.

14. The system of claim 10, wherein the communication channel is a wired or wireless communication channel.

15. The system of claim 13, wherein a 3-D data stream is generated from sensors at the remote patient location and the primary clinic location; and
wherein the 3-D data stream is used to establish the 3-D display for the doctor patient interaction.

16. The system of claim 13, wherein a 3-D data stream is generated from sensors at the remote patient location and the primary clinic location; and
wherein the 3-D data stream is used to establish the virtual reality environment for the doctor patient interaction.

17. A system comprising a remote patient location and a doctor's location at a primary clinic for development of an initial diagnosis and an initial treatment plan for a patient at the remote location, the system comprising:
the remote patient location and the doctor's location at the primary clinic;
the remote patient location and the doctor's location at the primary clinic comprising sensors that generate 3-D data streams of the volume of the locations using a processing power available at the locations;
an input and output (I/O) to accept inputs from a patient and provide outputs to the patient;
a natural language user input module for input from the patient via the I/O comprising the patient's current medical problems;
a language understanding module coupled to the user input module to convert the user inputs in natural language to medically understandable language;
an artificial intelligence (AI) with machine learning (ML) capability to generate questions to the patient to clarify the patient's problem;
an output to user-Questions in natural language presentation module coupled to the language understanding module to present the generated questions by the AI with ML capability to the patient in natural language;
a continuously updating conversational language data input into a continuously updating conversational database coupled to the language understanding module;
a stored language data base;
a learned questions and answers database;
a continuously updating clinical knowledge database;
an automatic diagnostic unit that uses the information in the databases and an understanding of the patient's current problem to generate a preliminary diagnosis and a preliminary treatment plan for the patient;
a communication link between the remote patient location and the doctor's location at the primary clinic, to send the preliminary diagnosis and the preliminary treatment plan to the doctors at the primary clinic location for review.

18. The system of claim 13, wherein the doctors at the clinic location are able to interact with the patient to further clarify and refine the diagnosis and the treatment plan.

19. The system of claim 18, wherein the patient and doctors interact as their respective avatars in a virtual environment.

20. The system of claim 18, wherein the patient and doctors interact over the network in the 3-D digital display.
